(19) United States
(12) Patent Application Publication
(10) Pub. No.: US 2018/0318538 A1
(43) Pub. Date: Nov. 8, 2018

(54) GUIDABLE INTUBATION STYLET

(71) Applicants: John William Nowlin, Austin, TX (US); John Howard Nowlin, Austin, TX (US)

(72) Inventors: John William Nowlin, Austin, TX (US); John Howard Nowlin, Austin, TX (US)

(21) Appl. No.: 15/772,764

(22) PCT Filed: Nov. 3, 2016
(86) PCT No.: PCT/US16/60329

§ 371 Date: May 1, 2018

Related U.S. Application Data
(60) Provisional application No. 62/250,109, filed on Nov. 3, 2015.
(60) Provisional application No. 62/359,585, filed on Jul. 7, 2016.

(51) Int. Cl. A61M 16/04; A61B 1/267

(52) U.S. Cl. CPC A61M 16/0488; A61M 2205/502; A61B 1/267

(57) ABSTRACT

Implementations described and claimed herein provide a guidable intubation stylet comprises a handle having a handle body. The handle body defines a cavity. An opening is disposed at a proximal end of the handle body, and a chamber is disposed at a distal end of the handle body. A joystick has a steering grip extending proximally from a joystick body. The joystick body is disposed in the cavity with the steering grip extending through the opening. A stylet shaft is connected to the handle body at the distal end, and a controllable tip is disposed distal to the stylet shaft. One or more wires are connected to the joystick body and extend distally through the chamber. A rotation of the joystick body using the steering grip causes a displacement of the one or more wires, and the displacement of the one or more wires moves the controllable tip.

### CROSS REFERENCE TO RELATED APPLICATIONS

The present application claims priority to U.S. Provisional Patent Application No. 62/250,109, filed Nov. 3, 2015, and U.S. Provisional Patent Application No. 62/359,585, filed Jul. 7, 2016. The contents of the above-mentioned patent applications are hereby incorporated by reference in their entirety.

### TECHNICAL FIELD

The presently disclosed technology relates generally to systems and methods for intubation in a trachea of a patient and more particularly to a guidable intubation stylet configured to facilitate intubation in infants, children, and other difficult contexts.

### BACKGROUND

Endotracheal intubation is generally performed by medical specialists (e.g., anesthesiologists, otolaryngologists, critical care specialists, etc.) to provide airway maintenance and protection for patients. For example, endotracheal tubes facilitate artificial intubation of unconscious or anesthetized patients, particularly during surgical procedures. Often, patients in pediatric intensive care units (ICUs) and neonatal ICUs have been intubated.

Endotracheal intubation is thus a common procedure typically performed with a non-video laryngoscope, such as a straight bladed Miller or curved bladed Macintosh and unstyleted endotracheal tube (“ETT”). However, anatomic variants in patients complicate such procedures. Intubating children with mandibular hypoplasia (i.e., small lower jaw) or many other craniofacial anomalies (e.g., cleft lip and palate, Pierre-Robin sequence, Crouzon syndrome, Treacher-Collins syndrome, Aperts syndrome, etc.) can be extremely difficult, if not impossible, with conventional techniques. Further exacerbating these challenges, infants, particularly premature infants, have a small size.

Video intubation laryngoscopes, such as glidescopes, facilitate management of pediatric airways amid these difficulties. These devices provide visualization of the larynx via a video camera placed at an angle on a tip of a laryngoscope blade. The video camera views the larynx forward of what can be seen looking down the axis of the laryngoscope blade. The image is typically displayed on a small video screen. While the larynx is visualized, however, these devices necessitate deployment of the ETT through the larynx and into the trachea along a nonlinear path.

To maneuver along the nonlinear path, many conventional systems and methods utilize semi-rigid stylets that are bent along an estimated curvature to reach the larynx prior to or following insertion into the ETT. The styletted ETT is then inserted into the oral cavity and advanced into the hypopharynx where it is visualized with the video intubation laryngoscope. If the estimated curvature of the stylet is correct, the ETT is inserted through the vocal cords and into the trachea. However, if the estimated curvature is incorrect, the ETT and stylet are withdrawn, and the stylet is adjusted to a new estimated curvature. This process is repeated until the estimated curvature is correct and the ETT is properly inserted into the larynx and trachea.

The trial and error of this process is often problematic during airway maintenance and protection, particularly in the context of pediatric patients. The pulmonary reserve in pediatric patients can be poor with their blood oxygen level falling rapidly when they are not being ventilated. Thus, the video laryngoscope is usually withdrawn between each attempt, and a bag and facemask are used to attempt to re-oxygenate the child. Because of the anatomy of these patients, bag and mask ventilation can be very difficult. Countless deaths or cases of brain damage due to anoxia have occurred in these situations. It is with these observations in mind, among others, that the presently disclosed technology was conceived and developed.

### BRIEF SUMMARY

The presently disclosed technology addresses the foregoing issues, among others, by providing systems and methods for airway maintenance and protection, including a guidable intubation stylet and methods of manufacturing and using the same. In one implementation, a guidable intubation stylet comprises a handle having a handle body. The handle body defines a cavity. An opening is disposed at a proximal end of the handle body, and a chamber is disposed at a distal end of the handle body. A joystick has a steering grip extending proximally from a joystick body. The joystick body is disposed in the cavity with the steering grip extending through the opening. A stylet shaft is connected to the handle body at the distal end, and a controllable tip is disposed distal to the stylet shaft. The stylet shaft and the controllable tip extend along a longitudinal axis. One or more wires are connected to the joystick body and extend distally through the chamber and along the longitudinal axis. A rotation of the joystick body using the steering grip causes a displacement of the one or more wires, and the displacement of the one or more wires moves the controllable tip.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE FIGURES

FIG. 1 is a longitudinal side view of an example guidable intubation stylet.

FIG. 2 shows the guidable intubation stylet of FIG. 1 with a portion of the handle and a stylet cover removed to show interior components.

FIGS. 3A and 3B are top and side views, respectively, of a first portion of an example handle.

FIGS. 4A and 4B illustrate a side view and a bottom view, respectively, of an example joystick with fixed connectors.

FIG. 5 shows a side view of another example joystick having adjustable connectors.

FIGS. 6A and 6B are side and bottom views, respectively, of the joystick of FIG. 5 with the adjustable connectors removed.

FIG. 7 depicts an example adjustable connector.

FIGS. 8A and 8B show a side view and a proximal end view, respectively, of an example stylet cover.

FIGS. 9A, 9B, and 9C illustrate top, front, and side views, respectively, of an example gimbal.

FIG. 10 depicts an example shaft.

FIGS. 11A and 11B show a proximal end view and side view, respectively, of an example handle connector.

FIGS. 12A and 12B are a detailed side view and a cross sectional view of an example stylet shaft.

FIGS. 13A and 13B show a detailed side view and a cross sectional view of another example stylet shaft comprising a shaped rod.

FIGS. 14A and 14B illustrate a detailed side view and a cross sectional view of another example stylet shaft comprising a coiled wire.

FIGS. 15A and 15B are a detailed side view and a cross sectional view of an example controllable tip.

FIGS. 16A and 16B show side and distal end views of a distal tip.

FIG. 17 illustrates another example controllable tip having jointed links, a transitional link, and a distal tip link.

FIGS. 18A and 18B depict an example transitional link.

FIGS. 19A-19C show an example jointed link.

FIGS. 20A-20D illustrate an example distal tip link.

FIG. 21 illustrates an example guidable intubation stylet inserted into an endotracheal tube (“ETT”).

FIG. 22 shows a sagittal cross sectional view of the stylet and ETT being inserted into a patient, the stylet and ETT shown positioned superior of the larynx.

FIG. 23 depicts the stylet maneuvering to position for insertion into the larynx.

FIG. 24 shows the stylet and ETT extending through the larynx into the trachea.

FIG. 25 illustrates example operations for performing a video laryngoscope endotracheal intubation using a guidable intubation stylet.

### DETAILED DESCRIPTION

Aspects of the present disclosure involve systems and methods for tracheal intubation using a guidable intubation stylet. The guidable intubation stylet facilitates deployment of an endotracheal tube (“ETT”) in challenging contexts, such as for pediatric patients, including infants and children, patients with anatomic variants, and/or the like. In one aspect, the guidable intubation stylet includes a controllable tip, a stylet shaft, and a handle. The controllable tip includes a series of joints which may be manipulated using one or more wires extending through the stylet shaft from the controllable tip to the handle. A joystick disposed in the handle displaces the one or more wires to control a curvature direction of the controllable tip, thereby providing precise control over an angle and degree of flex of the controllable tip. By controlling the curvature direction using the guidable intubation stylet, the ETT can be manipulated in situ for deployment along a curvature path through the larynx into the trachea without removal.

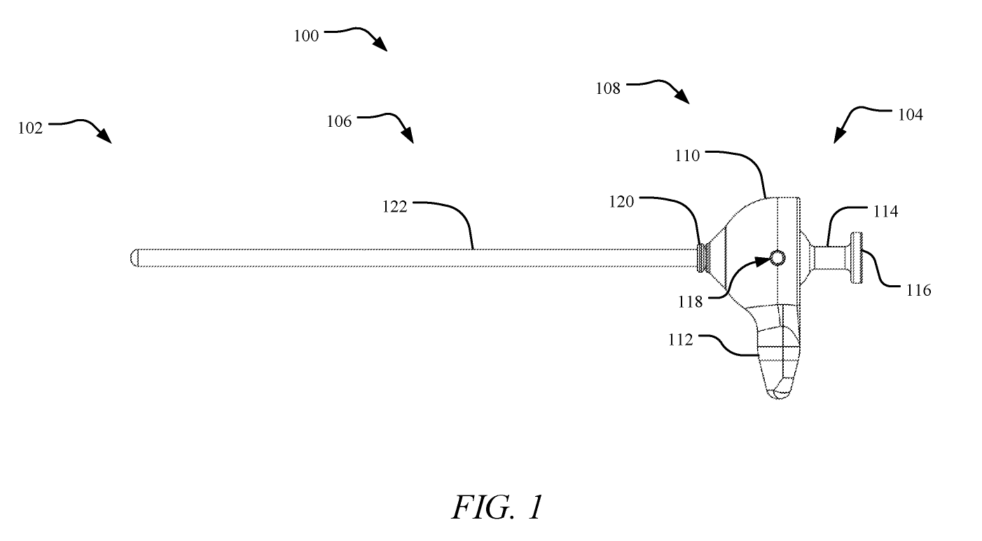

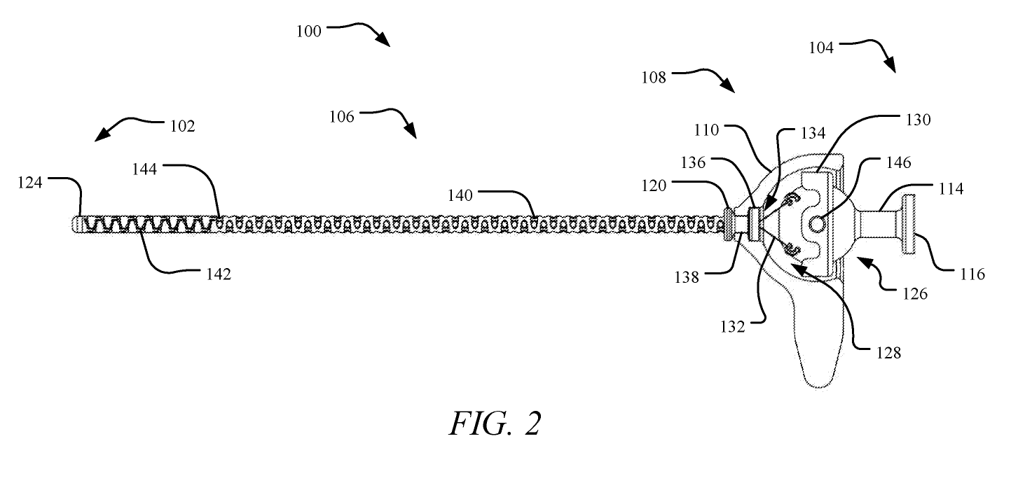

To begin a detailed description of an example guidable intubation stylet 100, reference is made to FIGS. 1-2. In one implementation, the guidable intubation stylet 100 extends between a distal end 102 and a proximal end 104. A handle 108 is disposed at the proximal end 104 with a stylet 106 extending distally therefrom.

The handle 108 includes a grip 112 protruding from a handle body 110. The grip 112 and the handle body 110 may be shaped to facilitate gripping by one hand while the other hand is available for other tasks. The hand that is holding the grip 112 may also be used for steering the stylet 106 using a steering grip extending proximally from the handle body 110. In one implementation, the steering grip includes an elongated body 114 extending proximally along a length until reaching a proximal grip 116 extending in a direction traverse to the length of the elongated body 114.

In one implementation, a joystick 126 includes the steering grip. The joystick 126 is disposed within a cavity 128 of the handle body 110 with the elongated body 114 extending proximally from the cavity 128. A gimbal 130 is connected to the joystick 126 providing a pivoted support permitting rotation about a single axis. More particularly, the handle body 110 includes a revolute joint 118 and the gimbal includes corresponding protrusions 146 along a revolute joint allowing the gimbal 130 to rotate freely along a single axis. One or more wires 132 are connected to the joystick 126, such that rotation of the joystick 126 using the steering grip displaces the wires 132.

The wires 132 extend distally through or otherwise along a length of the stylet 106. In one implementation, a handle connector mounts the stylet 106 to the handle body 110. The handle connector includes a distal end 120 connected to a proximal end 136 by an elongated portion 138. In one implementation, the distal end 120 is disposed outside the handle body 110 with the elongated portion 138 and the proximal end 136 disposed within the handle body 110. An opening 134 may extend through the handle connector from the proximal end 136 to the distal end 120 with a proximal end of the stylet 106 disposed in the opening 134 and the wires 132 extending distally through the opening 134 into one or more openings in the stylet 106.

In one implementation, the stylet 106 includes a stylet shaft 140, a controllable tip 142, and a distal tip 124, which may be covered with a stylet cover 122. The controllable tip 142 connects to the stylet shaft 140 at a transition 144, and the distal tip 124 extends distally from the controllable tip 142. The wires 132 extend through the stylet shaft 140 and connect to the distal tip 124 and/or the controllable tip 142. Thus, the controllable tip 142 may be maneuvered by rotating the joystick 126 to displace the wires 132.

The controllable tip 142 may include a series of joints, which the wires 132 may manipulate to move the controllable tip 142 in a selected direction. In one implementation, the controllable tip 142 includes a series of flexible joints, each disposed perpendicularly to an adjacent joint, connecting disks having one or more openings through which the wires 132 extend. Angled surfaces between each disk limit an amount of bending at each of the joints.

The joystick 126 displaces the wires 132 for movement of the controllable tip 142 to a precise angle and/or degree of flex. In one implementation, the wires 132 are secured at a distal end of the controllable tip 142, such that an application of tension on the wires 132 caused by actuation of the joystick 136 pulls the controllable tip 142 a desired amount. When the joystick 126 is moved in one direction, the wires 132 bend the controllable tip 142 along a curvature path accordingly. Returning the joystick 126 to center within the cavity 128 applies a tension on the opposite side of the controllable tip 142, causing the controllable tip 142 to straighten. The controllable tip 142 is thus bendable in a controlled manner in two different directions to create a curve.

The stylet 106 may be made from a variety of materials facilitating controlled movement of the controllable tip 142. For example, the stylet shaft 140 may be a flexible tube, and the joints of the controllable tip 142 may be solid hinges made from a flexible material. The stylet shaft 140 and the controllable tip 142 may be constructed from a single piece of flexible material or as separate pieces of flexible material. Further, the wires 132 may connect directly to the controllable tip 142. Alternatively, the wires 132 may connect to the distal tip 124. In some implementations, the distal tip 124 is integral with the controllable tip 142. In other implementations, the distal tip 124 and the controllable tip 142 are separate pieces.

The controlled flex of the controllable tip 142 manipulates a deployment path of the ETT in situ without removal. The guidable intubation stylet 100 thus facilitates deployment of the ETT along a curvature path through the larynx into the trachea, particularly in challenging contexts, such as for pediatric patients, including infants and children, patients with anatomic variants, and/or the like. Further, the stylet 106 has a narrow profile, facilitating deployment of the ETT in pediatric patients or other patients with smaller anatomy. As an example, the stylet 106 may have a narrow profile sized to fit inside a 2.5 mm ETT, as compared to conventional ETT's with inner diameters of 6 mm or larger.

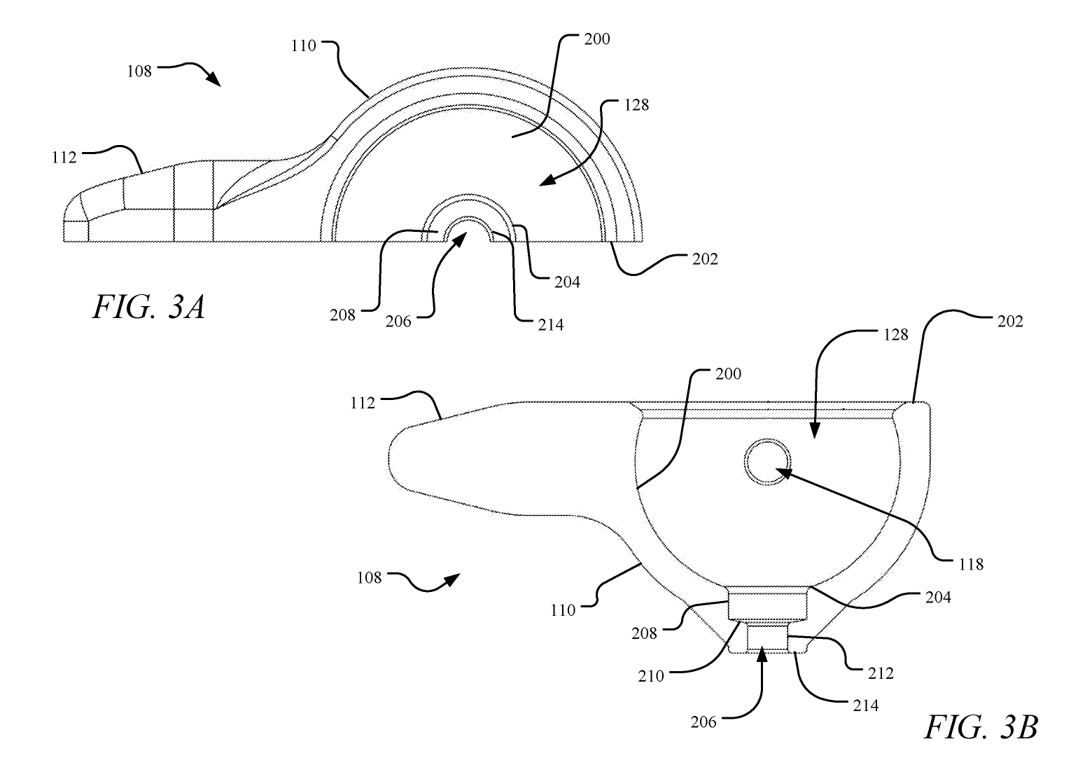

Turning to FIGS. 3A and 3B, a first portion of the handle 108 is shown. A second portion of the handle 108 may be substantially the same as the first portion. In one implementation, the handle 108 includes a cavity surface 200 extending along a contour from a proximal edge 202 to a distal edge 204. The cavity surface 200 forms the cavity 128 within which the joystick 126 and gimbal 130 are disposed and rotate. In one implementation, the cavity surface 200 forms the cavity 128 with a spherical shape. However, other shapes mirroring a shape of the joystick 126 are contemplated, such that the cavity surface 200 may include one or more contoured and/or angled surfaces.

In one implementation, proximal edge 202 has an opening extending into the cavity 128. The steering grip of the joystick 126 may extend through the opening for steering. The distal edge 204 similarly defines a chamber 206 through which the wires 132 extend into the stylet 106. The chamber 206 may be sized and shaped to engage the handle connector. In one implementation, the chamber 206 is formed by a proximal section 208 extending distally from the distal edge 204 of the cavity surface 200 to a ledge 210. The ledge 210 extends inwardly toward a center of the chamber 206 along an angle. A distal section 212 connects the ledge 210 to a distal edge 214 of the handle body 110. The proximal section 208 is configured to receive the proximal end 136 of the handle connector with the proximal end 136 abutting the ledge 210, and the elongated portion 136 extends along the distal section 212, with the distal end 120 disposed outside the handle body 110 past the distal edge 214. The wires 132 extend through the chamber 206 into the stylet shaft 140 to manipulate the controllable tip 142. The revolute joint 118 defined in the handle body 110 permits the gimbal 130 and thus the joystick 126 to rotate along an axis to provide movement of the controllable tip 142 with at least two degrees of freedom (e.g., up and down and side to side).

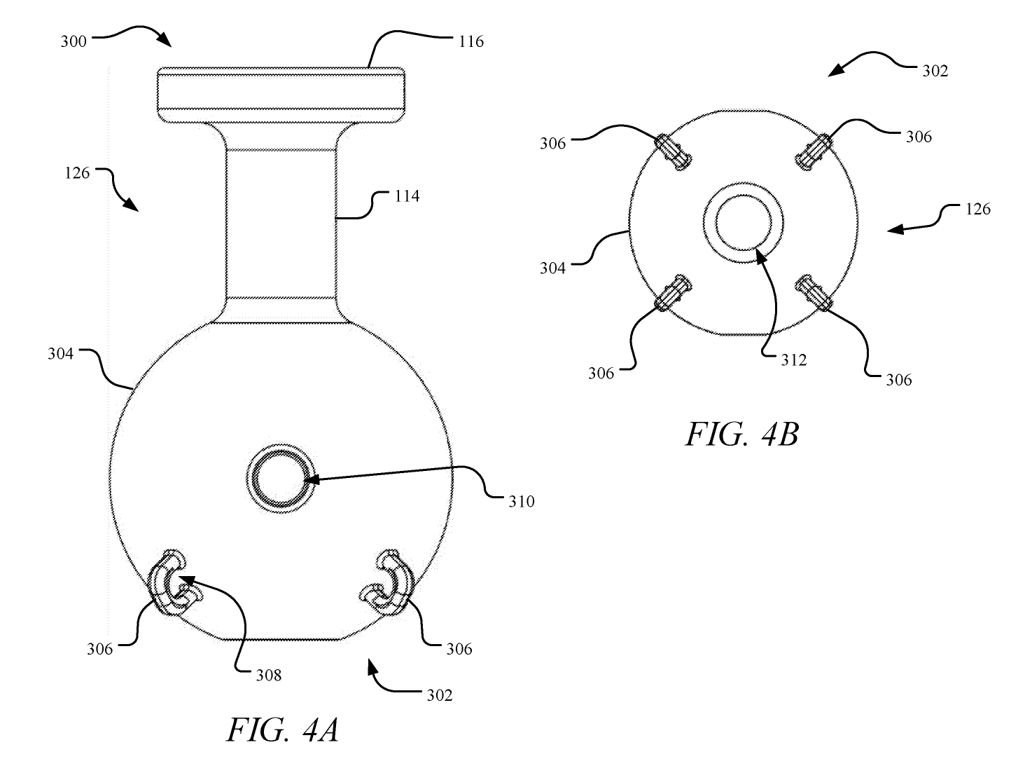

Referring to FIGS. 4A-4B, in one implementation, the joystick 126 extends between a proximal end 300 and a distal end 302. The steering grip may be disposed at the proximal end 300 with the elongated body 114 extending proximally from a joystick body 304. The joystick body 304 may have a variety of shapes and sizes configured to facilitate movement along a rotation path. For example, the joystick body 304 may have a rounded shape. In one implementation, the joystick body 304 includes a planar distal surface 312.

The proximal grip 116 of the steering grip provides a large surface area for rotating the joystick body 304. For example, the proximal grip 116 may have a surface area sized to facilitate manipulation using a thumb, finger, hand, and/or the like. The elongated body 114 of the steering grip increase a distance between the proximal grip 116 and a point of rotation of the joystick body 304, permitting a finer level of control.

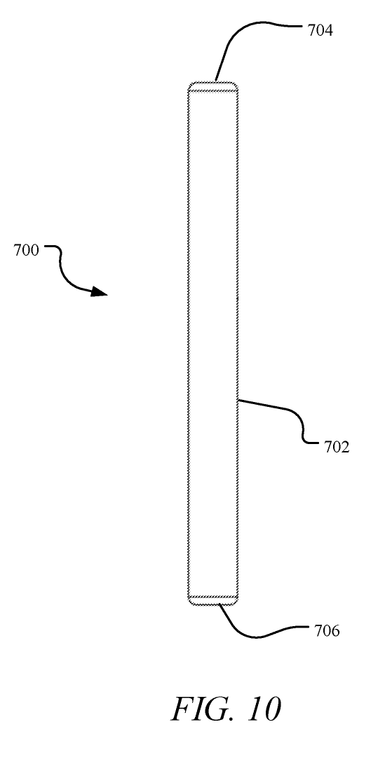

In one implementation, a joystick revolute joint 310 is defined in the joystick body 304. The joystick revolute joint 310 is configured to receive a shaft, such as the shaft 700 shown in FIG. 10. The shaft passes through the joystick revolute joint 310 extending through the joystick body 304 mounting the gimbal 130 to the joystick 126. As described herein, the gimbal 130 is a pivoted support permitting rotation of the joystick body 304 about a single axis on the rotation path to provide at least two degrees of freedom of movement of the stylet 106.

The rotation of the joystick body 304 displaces the wires 132 to move the controllable tip 142 along the two degrees of freedom of movement. In one implementation, one or more connectors 306 are disposed along the joystick body 304 to connect the wires 132 to the joystick body 304 to transfer the motion of the joystick 126 to the wires 132. The connectors 306 may each include an opening 308 configured to receive a corresponding wire 132. The connectors 306 may be disposed at equidistant locations around the joystick body 304 at the distal end 302.

As can be understood from FIGS. 4A-7, the connectors 306 may be fixed or adjustable. FIGS. 4A-4B show the joystick 126 with example fixed connectors 306. In one implementation, the connectors 306 include an arched body extending looping away from and back towards the joystick body 304 to form the opening 308. However, other forms of fixed connectors 306 are contemplated.

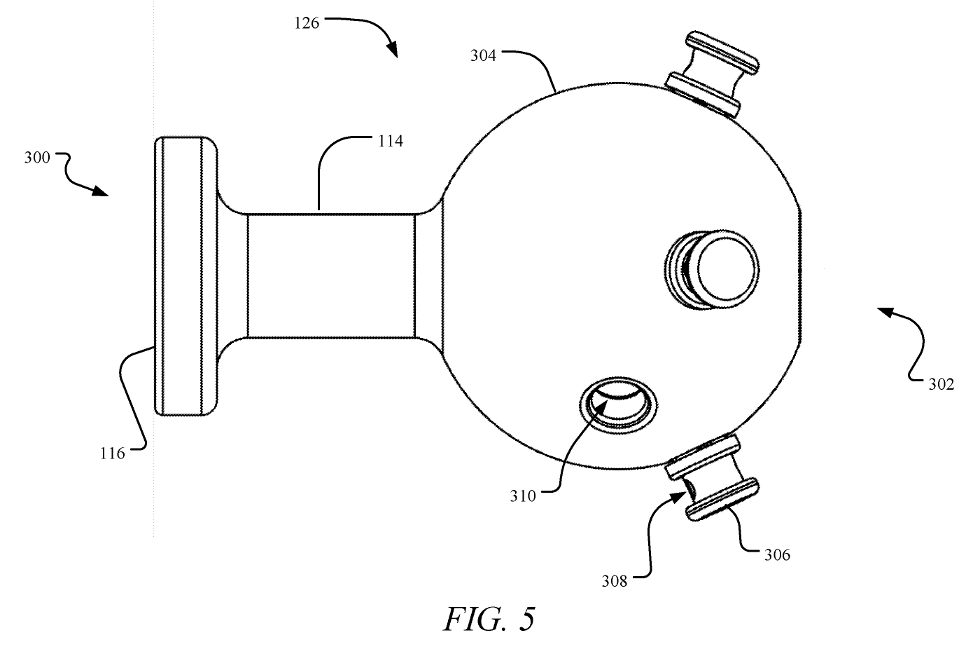

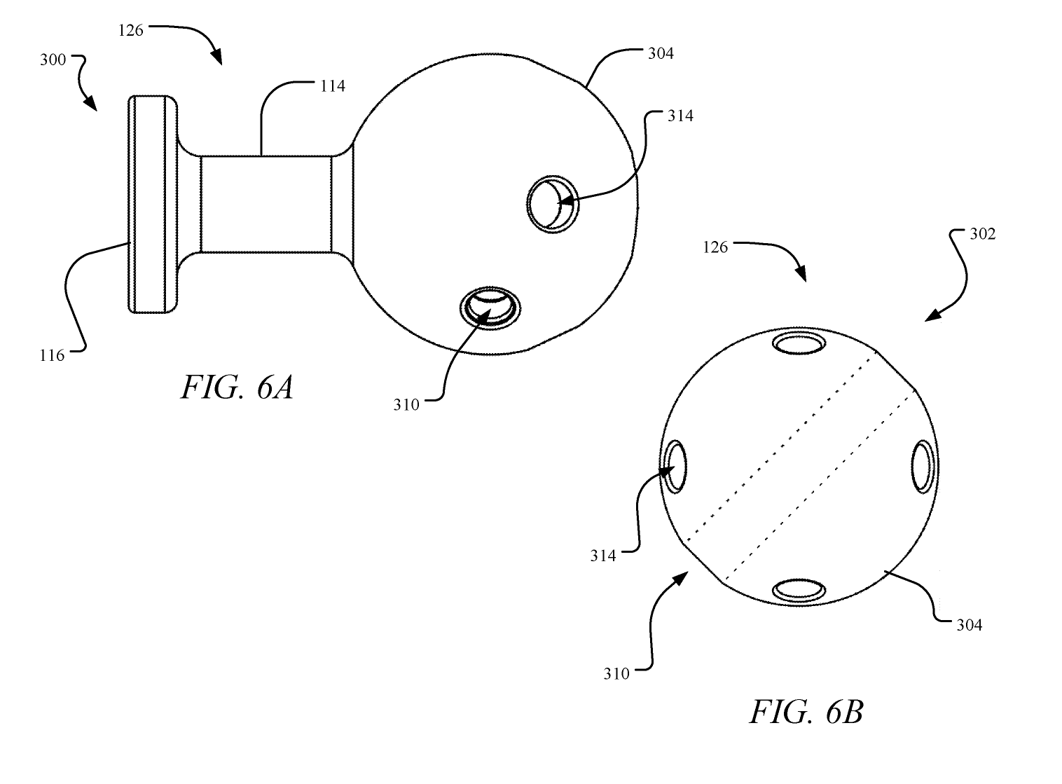

FIGS. 5-6B show the joystick 126 with example adjustable connectors 306. In one implementation, each of the adjustable connectors 306 are disposed within a corresponding connector opening 314. In other implementations, the adjustable connectors 306 are otherwise connected to or disposed on the joystick body 304.

A corresponding wire 132 may be attached to the adjustable connector 306 prior to adjusting a tension of the wire 132. Once all the wires 132 are connected to the adjustable connectors 306, the tension of the wires 132 may be adjusted, thereby providing a finer level of control over the degree of tension. Once the tension of the wires 132 is adjusted, the adjustable connectors 306 may be secured to the joystick body 304. The adjustable connectors 306 may be secured permanently or such that the wires 132 may be retensioned or otherwise adjusted.

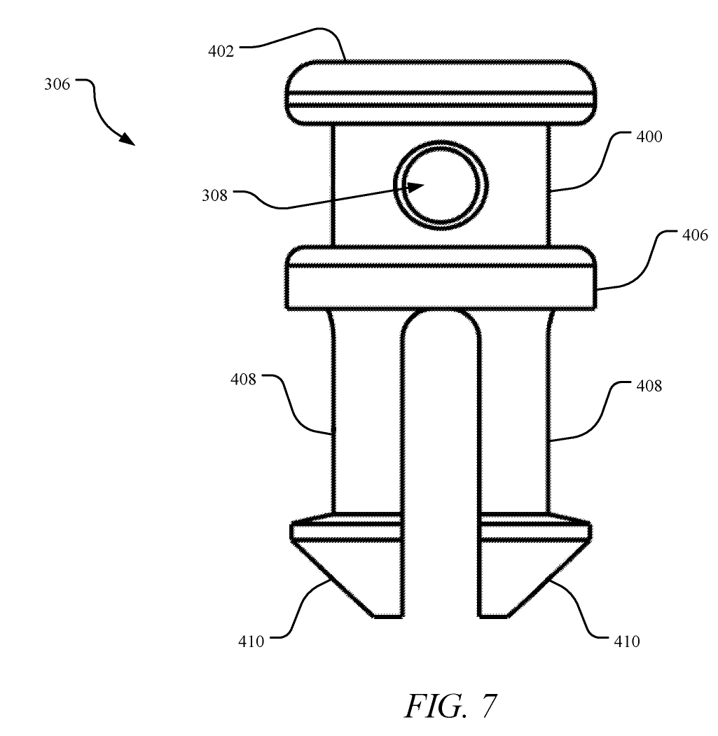

Turning to FIG. 7, an example adjustable connector 306 is shown. In one implementation, the adjustable connector includes a body 400 having the opening 308 extending therethrough and configured to engage a corresponding wire 132. The body 400 may have a variety of shapes, such as cylindrical. The body 400 extends between a proximal portion 402 and a distal portion 406. In one implementation, a set of legs 408 extend distally from the distal portion 406 and terminate in angled tips 410.

The set of legs 408 may be insertable into the connector opening 314 to engage the joystick body 304. In one implementation, the distal portion 402 is positioned against a surface of the joystick body 304 with the set of legs extending into the connector opening 314 and the angled tips 410 securing the adjustable connector 306 in place. It will be appreciated that the adjustable connector 306 may take a variety of forms and attach to the joystick body 304 in a various manners. For example, the adjustable connector 306 may be secured using physical methods, including, but not limited to, tension, pressure, and/or wedging. Moreover, the adjustable connector 306 may be threaded, screwed, or otherwise advanced into the connector openings 314. The adjustable connectors 306 may also be directly connected to the surface of the joystick body 304 using adhesives, welding, magnets, and/or other attachment methods.

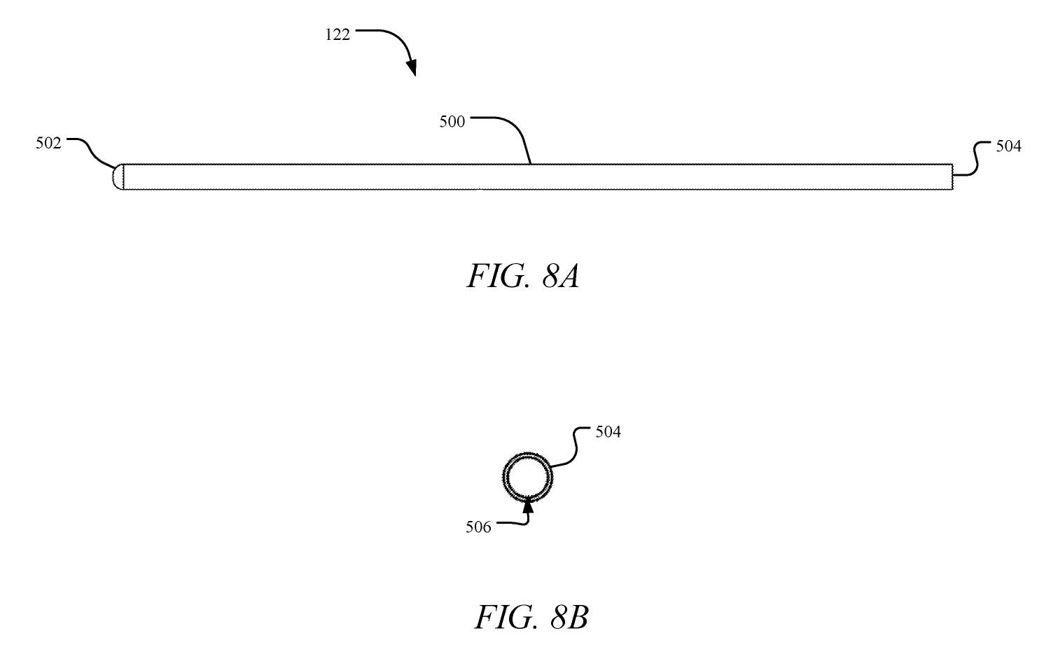

As can be understood from FIGS. 8A and 8B, the guidable intubation stylet 100 may include the stylet cover 122 to protect the stylet 106 during use. In one implementation, the stylet cover 122 includes an elongated tube 500 extending between a distal tip 502 and a proximal edge 504. The elongated tube 500 may have a thin wall defining a lumen 506 extending along the length of the elongated tube 500. The proximal edge 504 is open providing access to the lumen 506, and the distal tip 502 is closed. The stylet 106 may be positioned within the stylet cover 122, such that the stylet 106 is permitted to move freely within the lumen 506. The stylet cover 122 may connect to the handle 108 at the proximal edge 504. In one implementation, the proximal edge 504 connects to the handle at the handle connector.

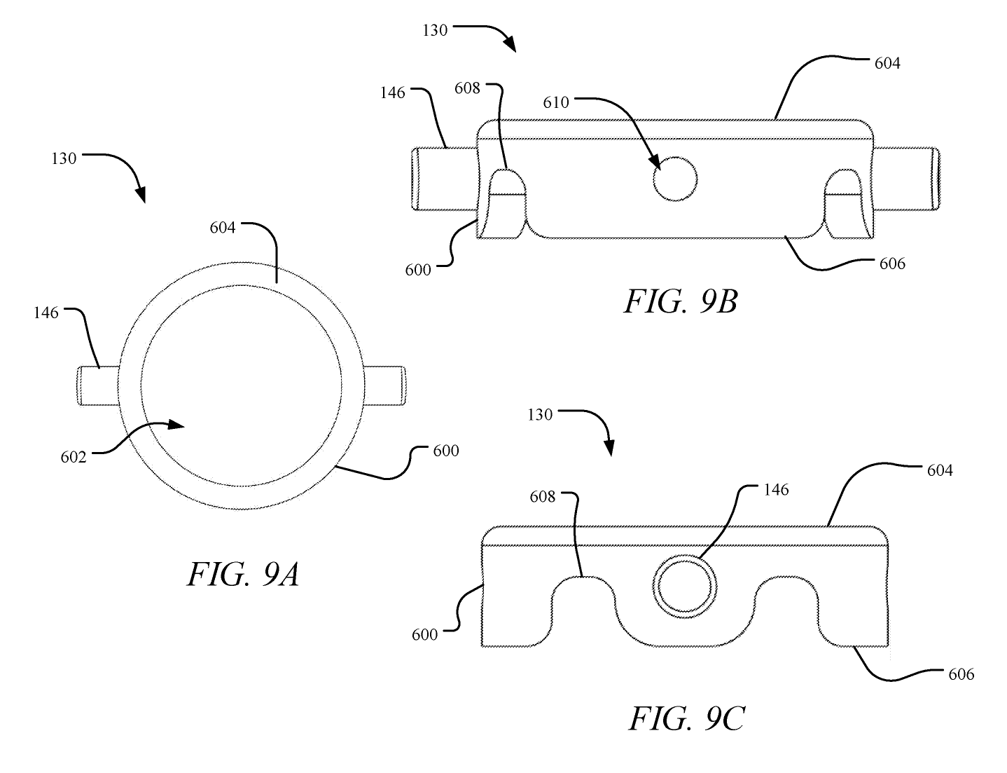

For a detailed description of an example of the gimbal 130, reference is made to FIGS. 9A-9C. In one implementation, the gimbal 130 includes a gimbal body 600 defining a gimbal opening 602. The gimbal body 600 extends between a proximal edge 604 and a distal edge 606. The gimbal body 600 may be sized and shaped to create the gimbal opening 602 with a size and shape mirroring a size and shape of the joystick body 304. For example, the gimbal body 600 may have a ring shape defining the gimbal opening 602 with a circular shape.

In one implementation, the gimbal 130 includes the protrusions 146 extending from the gimbal body 600 to connect to the revolute joint 118 of the handle body 110. A shaft opening 610 is defined in the gimbal body 600 corresponding to the to connect the joystick revolute joint 310 to mount the gimbal 130 to the joystick body 304. The joystick body 304 rotates along a single axis within the gimbal opening 602. The protrusions 146 connect the gimbal 130 to the revolute joint 118 of the handle body 110 permitting the gimbal body 600 to rotate along an axis perpendicular to an axis of rotation of the joystick body 304.

The gimbal body 600 may include one or more cutouts 608, which increase an extent of rotation of the joystick body 304 by providing extra space for the connectors 306 to rotate into. As such, the number, size, and shape of the cutouts 608 may mirror the configuration of the connectors 306 of the joystick 126.

As described herein, the gimbal body 600 includes a shaft opening 610 corresponding to the revolute joint 310 of the joystick body 304. As can be understood from FIG. 10, in one implementation, the shaft 700 may include a shaft body 702 extending from a proximal end 704 to a distal end 706. The shaft body 702 is inserted through the shaft opening 610 and the revolute joint 310 connecting the joystick body 304 to the gimbal body 600, such that the joystick body 304 is rotatable relative to the gimbal 600.

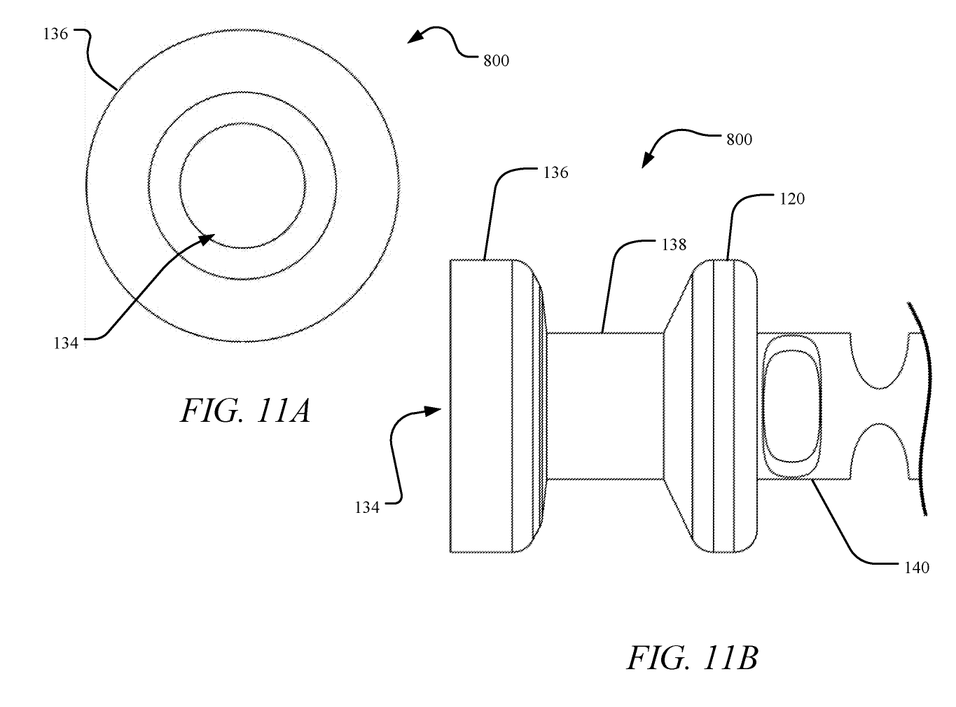

Turning to FIGS. 11A and 11B, detailed views of the handle connector 800 are shown. As described herein, the handle connector 800 connects the stylet shaft 140 to the handle body 110. The opening 134 is defined in the proximal end 136 and extends through the elongated portion 138 and the distal end 120. A proximal end of the stylet shaft 140 connects to the opening 134 to prevent rotation of the stylet 106 and thus misalignment of the controllable tip 142. The wires 132 pass through the opening 134 into the stylet shaft 140 to control movement of the controllable tip 142.

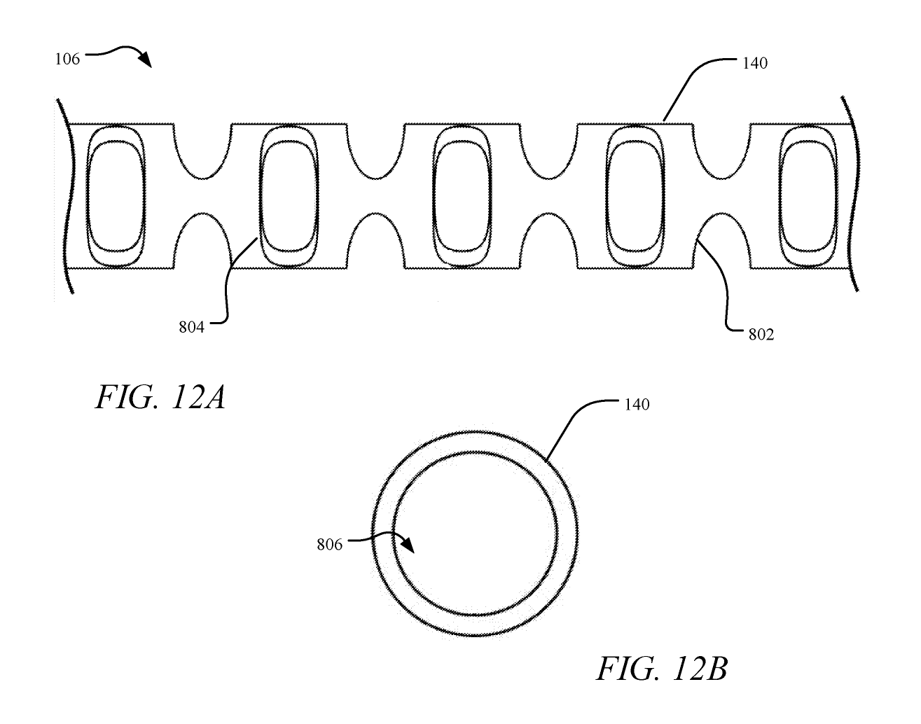

For a detailed description of examples of the stylet shaft 140, reference is made to FIGS. 12A-14B. Turning first to FIGS. 12A-12B, in one implementation, the stylet shaft 140 includes an internal opening 806 through which the wires 132 extend from the opening 134 of the handle connector 800. The internal opening 806 has a cross section profile that is sized to permit the wires 132 to move freely along a length of the stylet shaft 140. To increase flexibility of the stylet shaft 140 while maintaining the overall cross section profile, one or more peripheral cutouts 802 and/or one or more internal cutouts 804.

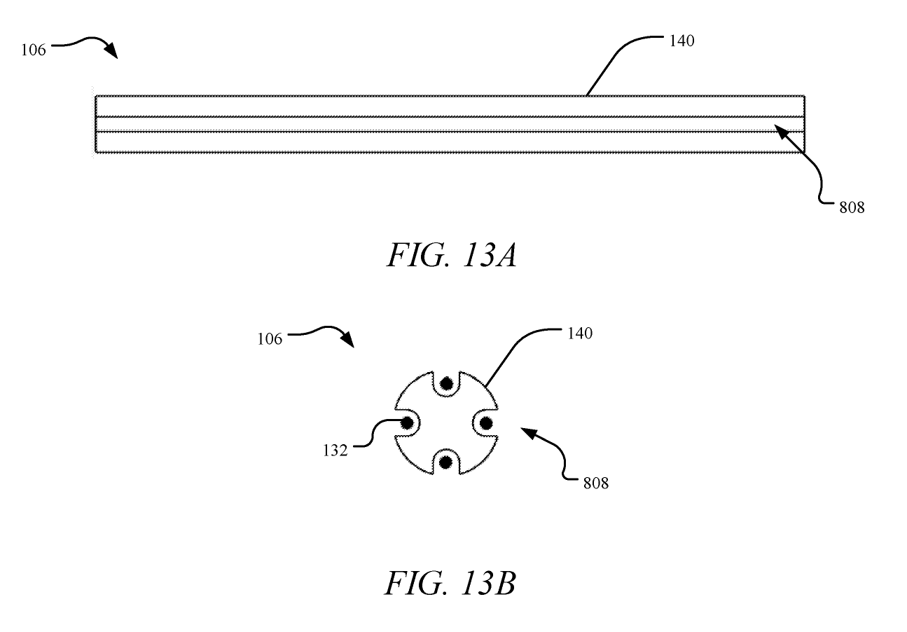

In another implementation shown in FIGS. 13A-13B, the stylet shaft 140 is a shaped rod with one or more channels 808 defined therein and extending along a length of the stylet shaft 140. Each of the wires 132 extends along a corresponding channel 808 such that it permitted to move freely along the length of the stylet shaft 140 within the channel 808. The channels 808 may be internal extending through the shaped rod and/or defined in a peripheral of the shaped rod. The shaped rod may be flexible, conform to the anatomy of the patient, and/or may be bendable into a curved shape where the stylet shaft 140 maintains the curved shape.

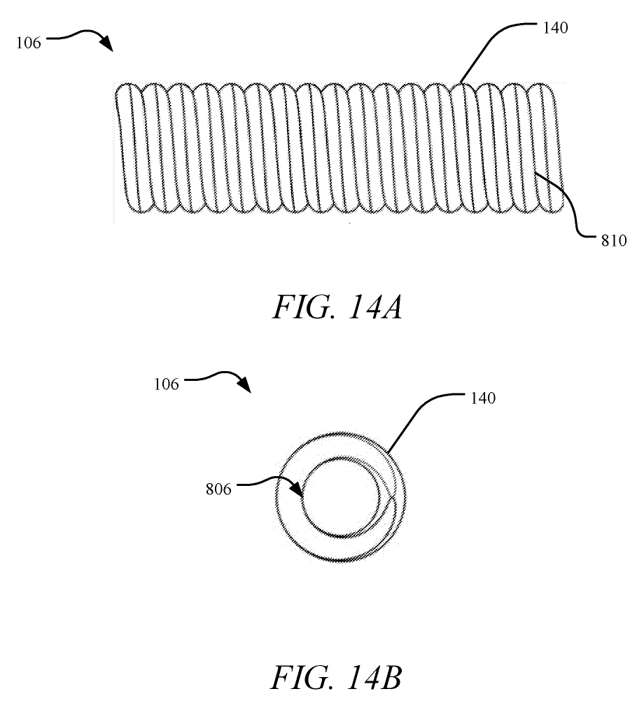

Turning to FIGS. 14A-14B, another implementation of the stylet shaft 140 is shown. The stylet shaft 140 includes a coiled wire 810 providing a structure of the stylet shaft 140 and defining the internal opening 806. The coiled wire 810 may be flexible, conform to the anatomy of the patient, and/or may be bendable into a curved shape, which may be held while maintaining the coiled structure.

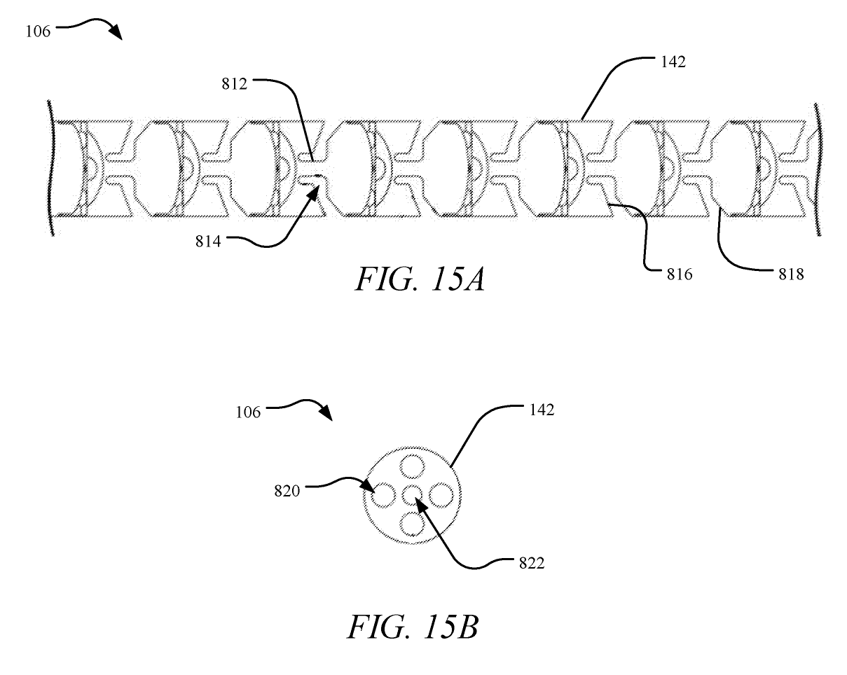

For a detailed description of an example of the controllable tip 142, reference is made to FIGS. 15A-15B. In one implementation, the controllable tip 142 includes a series of flexible joints 812 disposed along a central axis. Each of the flexible joints 812 is disposed in a perpendicular orientation relative to an adjacent flexible joint 812. Empty space 814 is formed along each of the flexible joints 812 to decrease the stress in the material of the flexible joints 812, thereby increasing the durability and flexibility of the flexible joints 812.

In one implementation, the controllable tip 142 includes distal flat surfaces 816 disposed relative to proximal flat surfaces 818. When the controllable tip 142 is bent the distal flat surfaces 816 press against the proximal flat surface 818, preventing the flexible joints 812 from extending beyond their operational limits and failing.

The controllable tip 142 includes one or more lumens 820, each configured to receive a corresponding wire 132. A number and layout of the lumens 820 mirrors an arrangement of the connection of the wires 132 to the joystick body 304 with the connectors 306. The wires 132 pass through the lumens 820 to pull the controllable tip 142 along a curvature path in a desired direction. In one implementation, a central lumen 822 is disposed along the central axis of the controllable tip 142, which may increase the flexibility of the controllable tip 142. Further, other devices or components may extend through or be passed through the central lumen 822.

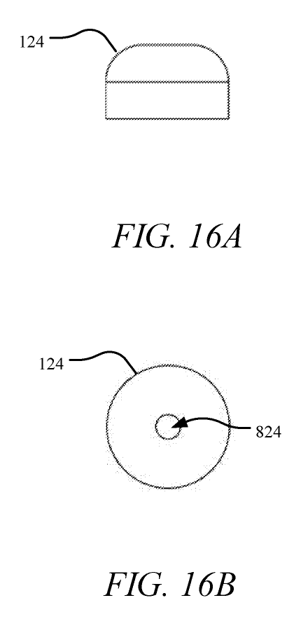

As shown in FIGS. 16A-16B, in one implementation, the wires 132 attach to the distal tip 124, which may be a separate piece attached to the controllable tip 142 or integrated into the controllable tip 142 with the wires connecting directly to the controllable tip 142. The distal tip 124 may include a central hole 824 disposed relative to the central lumen 822 of the controllable tip 142.

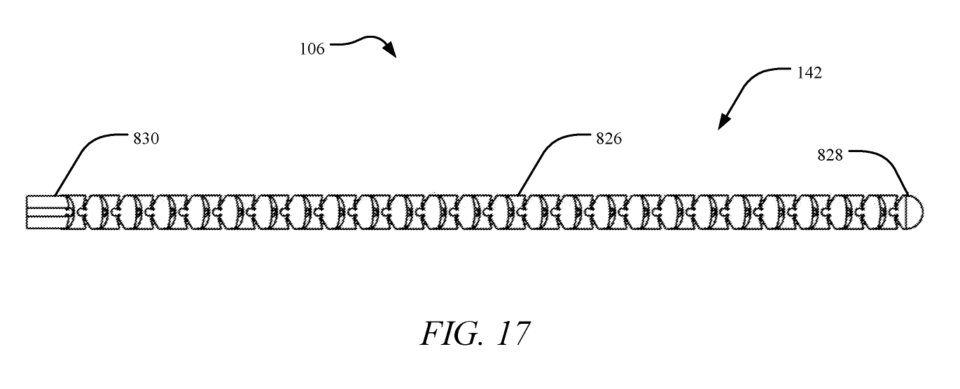

FIG. 17 illustrates another example of the controllable tip 142. In one implementation, the controllable tip 142 includes a series of jointed links 826 disposed along a central axis extending from a proximal end to a distal end. A distal tip link 828 may be disposed at the distal end to connect to the wires 132, and a transitional link 830 may be disposed at the proximal end connect the controllable tip 142 to the stylet shaft 140. In one implementation, the transitional link 830 forms the transition 144 of the stylet 106. The jointed links 826 permit the wires 132 to manipulate the controllable tip 142 in a desired direction.

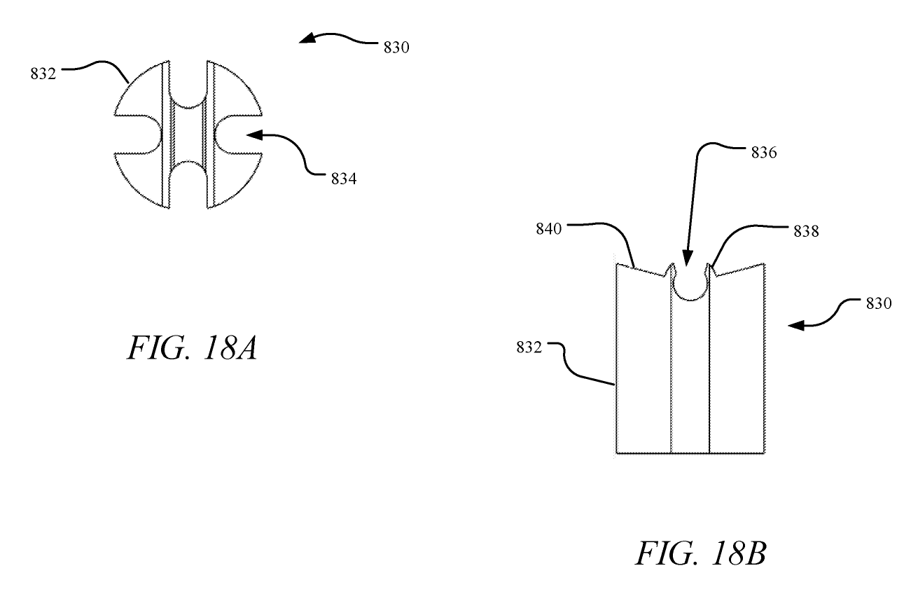

Turning to FIGS. 18A and 18B, the transitional link 830 connects the stylet shaft 140 to one of the jointed links 826. In one implementation, the transitional link 830 includes a body 832 with one or more slots 834 defined therein and extending along a length of the body 832. The slots 834 guide the wires 132 along the length. In one implementation, a hollow trough 836 is defined by a set of lips 838. The hollow trough 836 may have a variety of shapes, including without limitation, cylindrical. The hollow trough 836 acts as a portion of a hinge between the transitional link 830 and the jointed link 826, while the lips 838 enclose and limit the rotation of the jointed link 826. In one implementation, the transitional link 830 includes flat surfaces 840 configured to stop the jointed links 826 from bending past their operational limits.

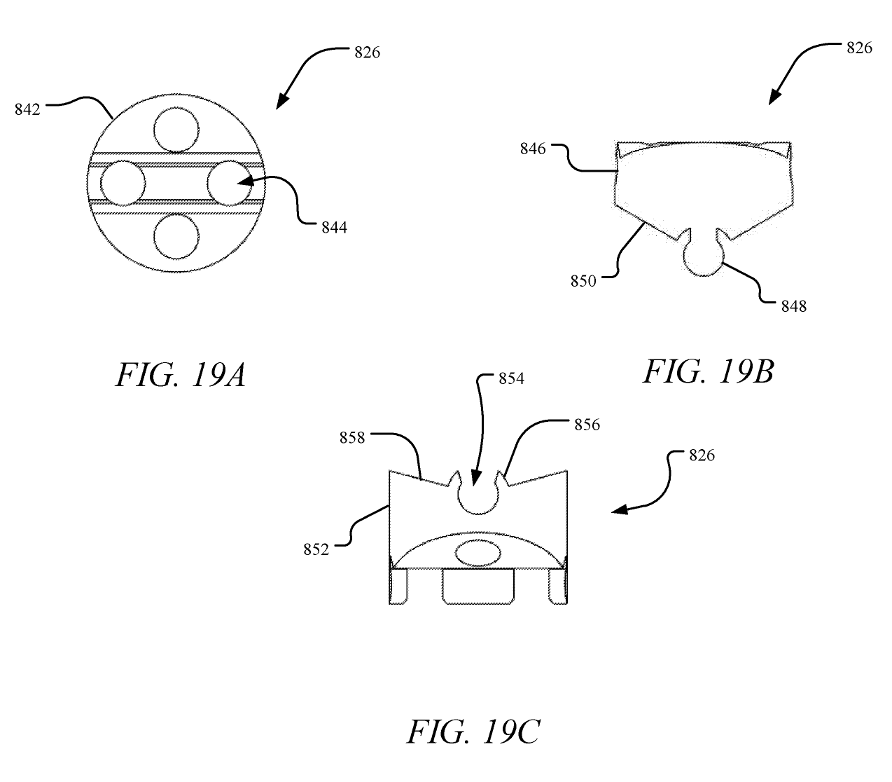

As shown in FIGS. 19A-19C, in one implementation, the jointed links 826 connect to each other to create the controllable tip 142 capable of bending in two directions in a controllable manner. The jointed links 826 include a body 842 with one or more lumens 844 defined therein through which the wires 132 extend. The wires 132 create tension and bending forces causing rotation on a knob 848 within a hollow trough 854 defined by a set of lips 856. A first flat edge 850 is disposed relative to a first longitudinal edge 846, and a second flat edge 858 is disposed relative to a second longitudinal edge 852. The first and second flat edges 850 and 858 limit the rotation of the knob 848 within the hollow trough 854 to prevent the jointed links 826 from bending past their operational limits. In one implementation, the knob 848 is disposed perpendicularly to the hollow trough 854 to alternative a direction of rotation of each of the jointed links 826. Stated differently, the jointed links 826 are each disposed perpendicularly to an adjacent jointed link 826. The lips 856 enclose the joints and in conjunction with the flat edges 850 and 858 limit the rotation of adjacent jointed links 826 when pressed together.

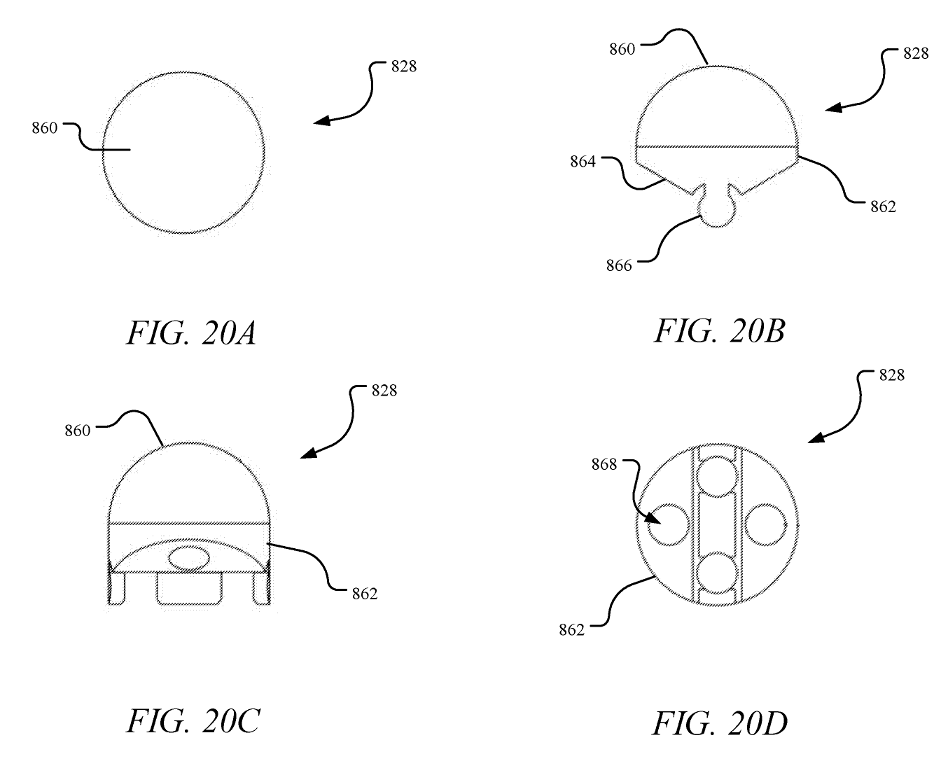

Referring to FIGS. 20A-20D, in one implementation, the distal tip link 828 includes a dome 860 with a rounded profile. The rounded profile of the dome 860 prevents the stylet 106 from puncturing the stylet cover 122 and/or causing injury to the patient during deployment. In one implementation, the distal tip link 828 includes a body 862 extending proximally from the dome 860. Flat surfaces 864 are defined in the body 862 with a knob 866 disposed therebetween. The knob 866 connects to the hollow trough 854 of one of the jointed links 826, providing rotation between the jointed link 826 adjacent to the distal tip link 828.

In one implementation, one or more holes 868 are defined in the body 862 through which corresponding wires 132 extend and connect to the distal tip link 828. Tension on the wires 132 pulls the jointed links 826 in two different directions to create a curve along the length of controllable tip 142. As described herein, the flat surfaces 864 prevent the distal tip link 828 from over-rotating.

The controllable tip 142 may be manufactured using a variety of techniques, such as additive manufacturing, which is a three dimensional (3D) printing press where layers of a raw material are added together or to other objects to build the material up into a shape. In one implementation, the controllable tip 142 is manufactured using stereolithography, which utilizes a controlled light source to selectively harden a photopolymer into the shape of the controllable tip 142. As described herein, the controllable tip 142 may be separate from or integral with the stylet shaft 140. The stylet shaft 140 and/or the handle connector 800 may be similarly manufactured using additive manufacturing.

Additive manufacturing further facilitates the creation of complex geometries on small scales. For example, the geometries of the jointed links 826, the distal tip link 828, and/or the transitional link 830 may be formed using additive manufacturing. Thus, the controllable tip 142 and the stylet shaft 140 may be manufactured with minimal waste and during a single process. Manufacturing the guidable intubation stylet 100 is thus inexpensive and less complex. Further, with the reduced manufacturing cost and complexity, the guidable intubation stylet 100 may be disposable, which simplifies sterilization of equipment during airway maintenance and protection.

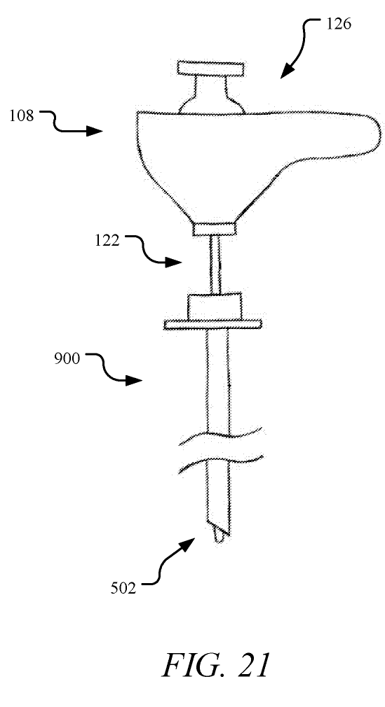

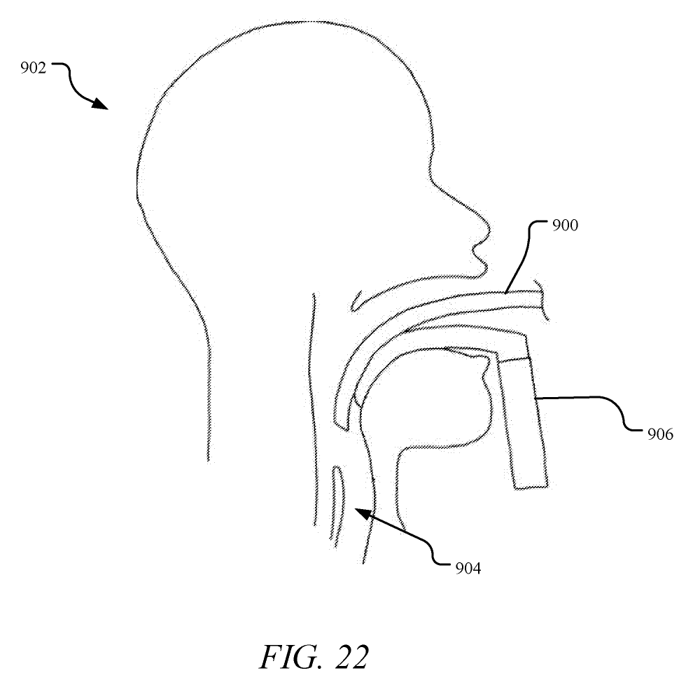

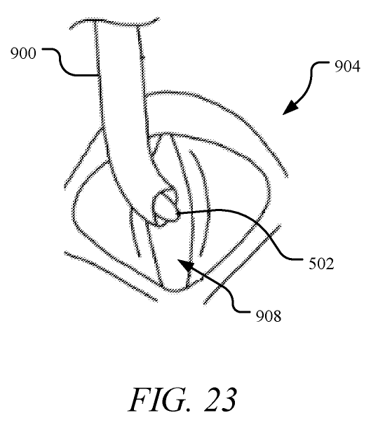

Referring to FIGS. 21-24, the guidable intubation stylet 100 may be used during a video laryngoscope endotracheal intubation. In one implementation, the guidable intubation stylet 100 is inserted into an ETT 900, as shown in FIG. 21. The ETT 900 with the inserted guidable intubation stylet 100 are inserted through a mouth of a patient 902 and advanced until superior of a larynx 908 of the patient 902, as shown in FIG. 22. Under visualization with an imaging system 906, such as a video laryngoscope, the ETT 900 and the inserted guidable intubation stylet 100 are guided into the larynx 908 using the joystick 126. FIG. 23 depicts the guidable intubation stylet 100 maneuvering to position the ETT 900 for insertion into the larynx 908.

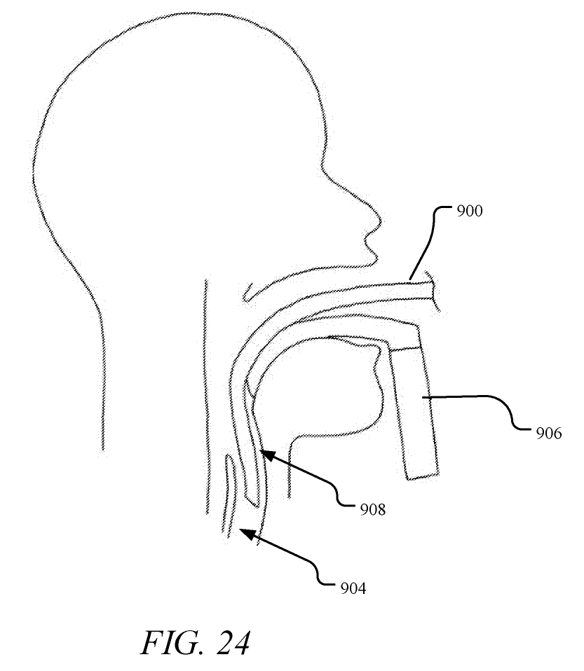

More particularly, conventional guidable stylets typically have only a single degree of freedom of movement (i.e., up and down). The guidable intubation stylet 100 includes the controllable tip 142 controllable with the joystick 126 displacing the wires 132. The guidable intubation stylet 100 thus provides at least two degrees of movement of the ETT 900 (e.g., up and down and side to side). By having the two degrees of freedom of movement, the need to rotate the ETT 900 during deployment is eliminated, thereby reducing trauma to the patient 902. The guidable intubation stylet 100 may be bent for inserting the ETT 900 into a trachea 904 of the patient 902. Once the controllable tip 142 is positioned near the larynx 908 as shown in FIG. 23, the ETT 900 may be maneuvered into the correct position for insertion into the trachea 904. As shown in FIG. 24, the ETT 900 is then inserted into the trachea 904, and the guidable intubation stylet 100 is removed, leaving the ETT 900 in place for airway maintenance and protection.

In one implementation, a fiber optic cable extends through the stylet shaft 140 and the controllable tip 142. The imaging system 906 is coupled with the fiber optic cable and positioned at the distal tip 124. A control device may be couple with the imaging system 906 to control an operation of the imaging system 906, for example, to control a field of view of the imaging system 906.

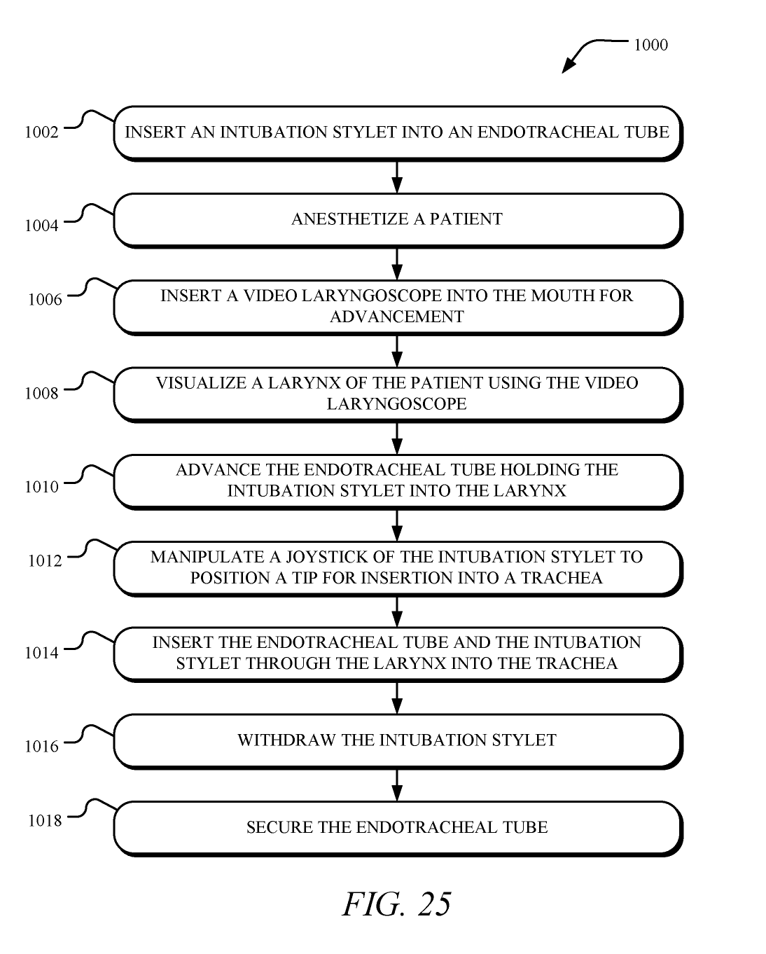

FIG. 25 illustrates example operations 1000 for performing a video laryngoscope endotracheal intubation using a guidable intubation stylet. In one implementation, an operation 1002 insets a guidable intubation stylet into an ETT, and an operation 1004 anesthetizes a patient. It will be appreciated that the operations 1002 and 1004 may be performed separately in either order or concurrently. Similarly, in some cases, the operation 1004 may not be performed. In one implementation, the operation 1004 anesthetizes the patient with a face mask and volatile anesthetic gas, via intravenous drugs, and/or in other manners.

In one implementation, an operation 1006 inserts a video laryngoscope into a mouth of the patient for advancement. The operation 1006 may advance the video laryngoscope with direct visualization. Once the video laryngoscope is positioned, an operation 1008 visualizes a larynx of the patient using the video laryngoscope. In one implementation, the larynx is visualized indirectly via a camera and display associated with the video laryngoscope. More particularly, the video laryngoscope may include a camera configured to capture images and/or video of the larynx and communicate them to the display for visualization. In one implementation, the video laryngoscope may be held with the non-dominant hand of the operator.

An operation 1010 advances the ETT holding the guidable intubation stylet into the larynx. In one implementation, the operation 1010 introduces the ETT and guidable intubation stylet under direct visualization of a side of the mouth corresponding to the dominant hand of the operator. The operation 1010 then advances the ETT and guidable intubation stylet until visualized on the display. The ETT and guidable intubation stylet are advances towards the larynx using the indirect visualization until a distal tip is positioned for insertion into the larynx.

In one implementation, an operation 1012 manipulates a joystick of the guidable intubation stylet to maneuver the distal tip is positioned for insertion through the larynx into the trachea. An operation 1014 inserts the ETT and the guidable intubation stylet through the larynx into the trachea. An operation 1016 withdraws the guidable intubation stylet, leaving the ETT in place, and an operation 1018 secures the ETT.

Various other modifications and additions can be made to the exemplary implementations discussed without departing from the spirit and scope of the presently disclosed technology. For example, while the embodiments described above refer to particular features, the scope of this disclosure also includes implementations having different combinations of features and implementations that do not include all of the described features. Accordingly, the scope of the presently disclosed technology is intended to embrace all such alternatives, modifications, and variations together with all equivalents thereof.

What is claimed is:

1. A guidable intubation stylet comprising:
a handle having a handle body, the handle body defining a cavity;
an opening disposed at a proximal end of the handle body;
a chamber disposed at a distal end of the handle body;
a joystick having a steering grip extending proximally from a joystick body, the joystick body disposed in the cavity with the steering grip extending through the opening;
a stylet shaft connected to the handle body at the distal end;
a controllable tip disposed distal to the stylet shaft, the stylet shaft and the controllable tip extending along a longitudinal axis; and
one or more wires connected to the joystick body and extending distally through the chamber and along the longitudinal axis, a rotation of the joystick body using the steering grip causing a displacement of the one or more wires, the displacement of the one or more wires moving the controllable tip.

2. The guidable intubation stylet of claim 1, wherein a gimbal is mounted to the joystick body and connected to the handle body within the cavity, the gimbal restricting the rotation of the joystick body along a rotational axis.

3. The guidable intubation stylet of claim 2, wherein the rotation of the joystick body along the rotational axis causes the displacement of the wires to move the controllable tip with a plurality of degrees of freedom of movement.

4. The guidable intubation stylet of claim 3, wherein the plurality of degrees of freedom of movement include at least one of up and down or side to side.

5. The guidable intubation stylet of claim 2, wherein the gimbal is mounted to the joystick body with a shaft.

6. The guidable intubation stylet of claim 1, wherein the stylet shaft is connected to the handle body with a handle connector at least partially positioned within the chamber.

7. The guidable intubation stylet of claim 1, wherein the steering grip includes an elongated body extending from the joystick body along a length until reaching a proximal grip, the proximal grip extending in a direction transverse to the length of the elongated body.

8. The guidable intubation stylet of claim 7, wherein the proximal grip has a surface area size to receive at least a thumb of an operator for steering.

9. The guidable intubation stylet of claim 1, wherein each of the one or more wires is connected to the joystick body with a connector.

10. The guidable intubation stylet of claim 9, wherein the connector is a fixed connector.

11. The guidable intubation stylet of claim 9, wherein the connector is an adjustable connector.

12. The guidable intubation stylet of claim 11, wherein the adjustable connector includes a body having an opening configured to engage a corresponding wire, the adjustable connector further including a set of legs configured to engage the joystick body in a connector opening.

13. The guidable intubation stylet of claim 1, wherein the stylet shaft includes an internal opening through which the wires extend and are permitted to move freely.

14. The guidable intubation stylet of claim 1, wherein the stylet shaft is a shaped rod with one or more channels each configured to receive a corresponding wire.

15. The guidable intubation stylet of claim 1, wherein the stylet shaft is a coiled wire.

16. The guidable intubation stylet of claim 1, wherein the controllable tip includes a series of flexible joints disposed along the longitudinal axis.

17. The guidable intubation stylet of claim 16, wherein each of the flexible joints is disposed perpendicularly relative an adjacent flexible joint.

18. The guidable intubation stylet of claim 16, wherein empty space is formed along each of the flexible joints to increase a flexibility of the controllable tip.

19. The guidable intubation stylet of claim 16, wherein the controllable tip includes a series of flat surfaces, each configured to meet a corresponding flat surface to prevent the flexible joints from extending beyond an operational limit.

20. The guidable intubation stylet of claim 1, wherein the one or more wires are connected to a distal tip connected to the controllable tip or directly to the controllable tip.
